(19) United States
(12) Patent Application Publication
(10) Pub. No.: US 2024/0209132 A1
(43) Pub. Date: Jun. 27, 2024

(54) COUPLING OF 2,3,5-TRIMETHYLHYDROQUINONE AND UNSATURATED ALCOHOLS

(71) Applicant: DSM IP ASSETS B.V., Heerlen (NL)

(72) Inventors: Werner BONRATH, Kaiseraugst (CH); Rolf KUENZI, Kaiseraugst (CH); Thomas NETSCHER, Kaiseraugst (CH)

(21) Appl. No.: 18/556,213

(22) PCT Filed: Apr. 26, 2022
(86) PCT No.: PCT/EP2022/061093

§ 371 Date: Oct. 19, 2023

(30) Foreign Application Priority Data
Apr. 28, 2021 (EP) 21170909.2

(51) Int. Cl. C08F 232/06; C08F 2/06; C08F 4/32; C08F 4/44

(52) U.S. Cl. CPC C08F 232/06; C08F 2/06; C08F 4/32; C08F 4/44

(57) ABSTRACT

The present invention relates to the formation of compound of the formula (I) by reacting 2,3,6-trimethylhydroquinone or a protected derivative thereof with the unsaturated alcohol of formula (IIIa) or (IIIb) in the presence of Gd(OTf)₃ or Tm(OTf)₃ or Al(OTf)₃ or Y(OTf)₃ or Fe(OTf)₂ or camphorsulfonic acid or BiCl₃ as acidic catalyst.

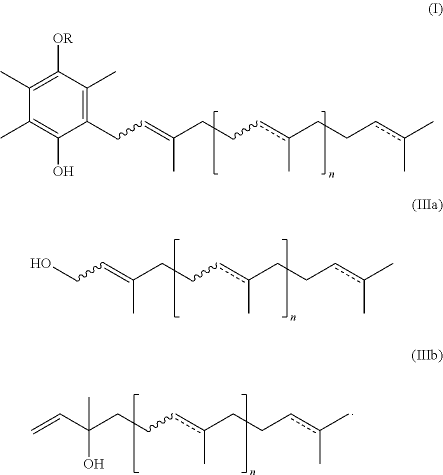

### TECHNICAL FIELD

The present invention relates to the field of the synthesis of chromanes and chromenes, particularly, of 3,4-dehydrotocotrienols, tocotrienols and tocopherols.

### BACKGROUND OF THE INVENTION

Chromane compounds comprising olefinic carbon-carbon double bonds are an important class of chemicals. Particularly, α-tocotrienol is an highly important member of this class.

The corresponding compound having a saturated side chain, i.e. α-tocopherol, is known to be prepared from 2,3,6-trimethylhydroquinone and isophytol in the presence of Lewis and/or Brønsted acids.

For example, WO 2004/063182 A1 discloses the formation of α-tocopheryl acetate from 2,3,6-trimethylhydroquinone-1-acetate and isophytol in the presence of a large variety of metal triflates. Isophythol has only one carbon-carbon double bond which is used for the coupling reaction, however, has no further double bond(s) which might lead to secondary ring formations, i.e. ring formation in the side chain.

WO 2004/046126 A1 discloses the formation of α-tocopheryl acetate from 2,3,6-trimethylhydroquinone-1-acetate and isophytol in the presence of a large variety of sulfonic acids, particularly triflic acid or p-toluenesulfonic acid. As isophythol is used, no secondary ring formation is possible.

EP 949255 A1 discloses the coupling of TMHQ with isophythol using sulfuric acid or a few sulfonic acids, particularly triflic acid or p-toluenesulfonic acid.

However, it has been shown that the use of those acidic catalysts are generally not suitable for the reaction of 2,3,6-trimethylhydroquinone (TMHQ) or its protected form with the respective unsaturated alcohols, such as geranylgeraniol or farnesol, because undesired cyclic compounds are formed.

This finding is completely in line with Kabbe and Heitzer, Synthesis 1978, 12, 888-889, who has reported that using the known synthetic pathway for vitamin E (i.e. α-tocopherol) from TMHQ and isophytol is not suitable for the synthesis of tocotrienols (i.e. from TMHQ and geranyllinalool) because the isoprenoid side chain undergoes acid-catalyzed secondary ring closure reactions.

The synthesis of tocotrienol and its precursors, however, is still of great importance.

### SUMMARY OF THE INVENTION

Therefore, the problem to be solved by the present invention is to find a suitable process for the manufacturing of the compound of the formula (I) from TMHQ or its protected form with unsaturated alcohols of the formula (IIIa) or (IIIb).

It has been very surprising that despite the teaching of the prior art, a few members of the metal triflates and sulfonic acids are suitable catalysts for this reaction and yield the compound of formula (I) in high yield and selectivity, particularly with low tendency of forming cyclic compounds by the isoprenoid side chains as side products.

Further aspects of the invention are subject of further independent claims. Particularly preferred embodiments are subject of dependent claims.

### DETAILED DESCRIPTION OF THE INVENTION

In a first aspect, the present invention relates to a process of manufacturing the compound of the formula (I)

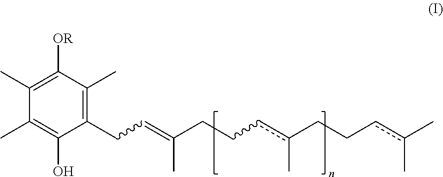

comprising a step of reacting a compound of the formula (II) with a compound of the formula (IIIa) or (IIIb)

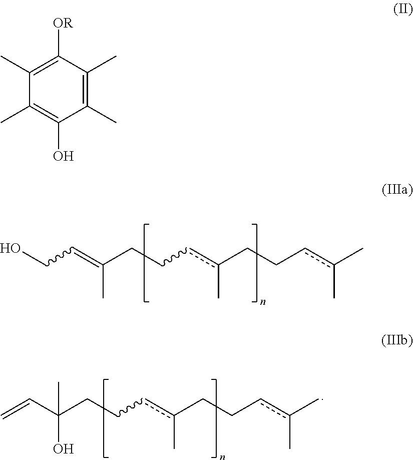

in the presence of an acidic catalyst being
either
   Gd(OTf)₃ or Tm(OTf)₃ or Al(OTf)₃ or Y(OTf)₃ or Fe(OTf)₂
or
   camphorsulfonic acid;
or
   BiCl₃;
wherein
   n=0 or 1 or 2 or 3 or 4 or 5 or 6 or 7 or 8 or 9 or 10 or 11 or 12;
   R represents hydrogen or R′ which is a phenol protecting group;
   OTf represents trifluoromethanesulfonate;
and
any bond having dotted line ( [symbol] ) represents independently from each other either a carbon-carbon single bond or a carbon-carbon double bond, with the proviso that at least one of the bonds having dotted lines represents a carbon-carbon double bond;
any wavy line represents independently from each other a carbon-carbon bond and which when linked to the carbon-carbon double bond is either in the Z- or in the E-configuration.

It is preferred, that any wavy lines when linked to a carbon-carbon double bond is in the E-configuration.

For sake of clarity, some terms used in the present document are defined as follows:

In the present document, a “C_{x-y}-alkyl” group is an alkyl group comprising x to y carbon atoms, i.e., for example, a C₁₋₃-alkyl group is an alkyl group comprising 1 to 3 carbon atoms. The alkyl group can be linear or branched. For example —CH(CH₃)—CH₂—CH₃ is considered as a C₄-alkyl group.

An “aralkyl” group is an alkyl group which is substituted by an aryl group.

Accordingly, in the present document, a “C_{x-y}-aralkyl” group is an aralkyl group comprising x to y carbon atoms, i.e., for example, a C₇₋₁₆-aralkyl group is an aralkyl group comprising 7 to 16 carbon atoms. The aralkyl group can be linear or branched. For example, benzyl group (—CH₂-C₆H₅) is considered as a C₇-aralkyl group.

In case identical labels for symbols or groups are present in several formulae, in the present document, the definition of said group or symbol made in the context of one specific formula applies also to other formulae which comprises the same said label.

The term “independently from each other” in this document means, in the context of substituents, moieties, or groups, that identically designated substituents, moieties, or groups can occur simultaneously with a different meaning in the same molecule.

In the present document, any dotted line in formulae represents the bond by which a substituent is bound to the rest of a molecule.

In the present document any bond having dotted line ( [symbol] ) in a chemical formula represents independently from each other either a carbon-carbon single bond or a carbon-carbon double bond.

Any wavy line in any formula of this document represents a carbon-carbon bond and which when linked to the carbon-carbon double bond is either in the Z or in the E-configuration. It is preferred in all molecules that the carbon-carbon double bond is in the E-configuration.

The term “OTf”, as used in this document, means trifluoromethanesulfonate.

The “pKₐ” is commonly known as negative decadic logarithm of the acid dissociation constant (pKₐ=−log₁₀ Kₐ). When the organic acid has several protons the pKₐ as used in this document relates to the dissociation constant of the last proton. For example, for a base having two basic sites the “pka” relates to pKₐ₂. The pKₐ are measured at standard temperature and pressure.

### Compound of the Formula (II)

The compound of formula (II) is either 2,3,5-trimethyl hydroquinone (=2,3,5-trimethylbenzene-1,4-diol, TMHQ) (R═H) or the mono-protected derivative thereof (R≠H; R═R′).

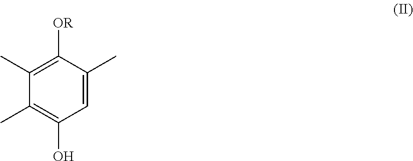

A phenol protecting group is a group which protects the phenolic group (OH) in any of the formulae in this document having R═H in said formulae and the protecting group can be easily removed, i.e. by state-of-the-art methods, resulting to the respective compound with the free phenolic group again.

The phenol protecting group R′ is introduced by a chemical reaction of the compound of the respective formula having H as R with a protecting agent.

The protecting agents leading to the corresponding phenol protecting groups are known to the person skilled in the art, as well as the chemical process and conditions for this reaction. If, for example, the phenol protecting group forms with the rest of the molecule an ester, the suitable protecting agent is for example an acid, an anhydride, or an acyl halide.

The phenol protecting group R′ is particularly selected from the groups consisting of

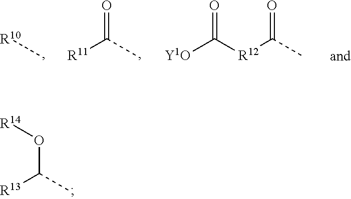

wherein R¹⁰ and R¹¹ represent independently from each other a C₁₋₁₅-alkyl or a fluorinated C₁₋₁₅-alkyl or a C₁₋₁₅-cycloalkyl or a C₇₋₁₅-aralkyl group;
R¹² represents a C₁₋₁₅-alkylene or a C₆₋₁₅-alkylene group;
and wherein either
   R¹³ represents a C₁₋₁₅-alkyl group or an alkyleneoxyalkyl group or a polyoxyalkylene group;
   R¹⁴ represents hydrogen or a C₁₋₁₅-alkyl group;
or
   R¹³ and R¹⁴ represent together a C₃₋₇-alkylene group forming a 5 to 7 membered ring;
and wherein the single dotted line represents the bond by which said substituent is bound to the rest of a molecule.

If R′ is equal to R¹⁰, the respective compound is an ether, which can be formed by the reaction of the respective protecting agent with the phenolic group (OH). In this case, the protecting agent may be for example an alkylation agent such as the respective C₁₋₁₅-alkyl or fluorinated C₁₋₁₅-alkyl or C₁₋₁₅-cycloalkyl or C₇₋₁₅-aralkyl halide, particularly iodide.

In one of the preferred embodiments R¹⁰ is a methyl group.

In another preferred embodiment R¹⁰ is a C₇₋₁₅-aralkyl group, preferably a benzyl group or a substituted benzyl group, particularly preferred a benzyl group.

If R′ is represented by

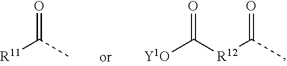

the respective compound is an ester of a carboxylic acid or dicarboxylic acid, which can be formed by the reaction of the respective protecting agent with the phenolic group (OH). In this case, the protecting agent may be for example an anhydride or halide of the respective carboxylic acid (1) or dicarboxylic acid (2).

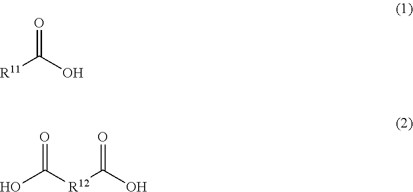

If the compound of the respective formula is an ester of a carboxylic acid or dicarboxylic acid, it is preferred that R′ is an C₁₋₇-acyl, preferably acetyl, trifluoroacetyl, propionyl or benzoyl group, or a substituted benzoyl group.

If R′ is

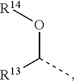

the respective compound is an acetal, which can be formed by the reaction of the respective protecting agent with the phenolic group (OH). In this case, the protecting agent may be for example, a respective aldehyde, alkyl halide, e.g. MeO(CH₂)₂OCH₂Cl, or an enol ether, e.g. 3,4-dihydro-2H-pyran.

In this case, the substituent R′ is preferably

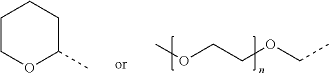

with n=0 or 1.

In some instances, acetals are also called “ethers”, particularly in the cases mentioned above: methoxymethyl ether (MOM-ether), β-methoxyethoxy-methyl ether (MEM-ether) or tetrahydropyranyl ether (THP-ether).

In another preferred embodiment, the respective compound is an ester of phosphoric acid, pyrophosphoric acid, phosphorous acid, sulphuric acid or sulphurous acid.

Depending on the reaction conditions, the esterification is either complete or partial, leaving some residual acid groups of the respective acid non-esterified.

It is most preferred that the protecting group R′ is a benzoyl group or a C₁₋₄-acyl group, particularly acetyl or trifluoroacetyl group, more particularly acetyl group. The molecules in which R′ represents an acyl group, particularly an acetyl group, can be easily prepared from the corresponding unprotected molecule by esterification, and the unprotected phenolic compound can be obtained from the corresponding ester by ester hydrolysis.

The phenol protecting group can be cleaved by a deprotecting reaction step b), as discussed later.

It is preferred that R′ is a phenol protecting group of the formula

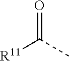

wherein R¹¹ is a C₁₋₁₅-alkyl or a fluorinated C₁₋₁₅-alkyl or a C₁₋₁₅-cycloalkyl or a C₇₋₁₅-aralkyl group, preferably a methyl or a benzyl group.

### Compound of the Formula (IIIa) or (IIIb)

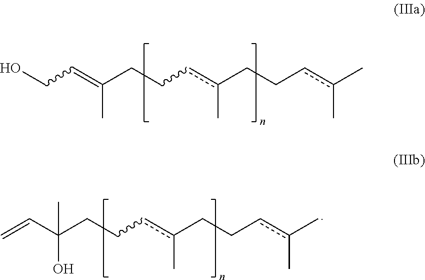 [0069] n=0 or 1 or 2 or 3 or 4 or 5 or 6 or 7 or 8 or 9 or 10 or 11 or 12.

It is preferred that n=0 or 1 or 2, preferably 1 or 2, most preferably n=2.

Examples of preferred compounds of the formula (IIIa) are compounds selected from the group consisting of

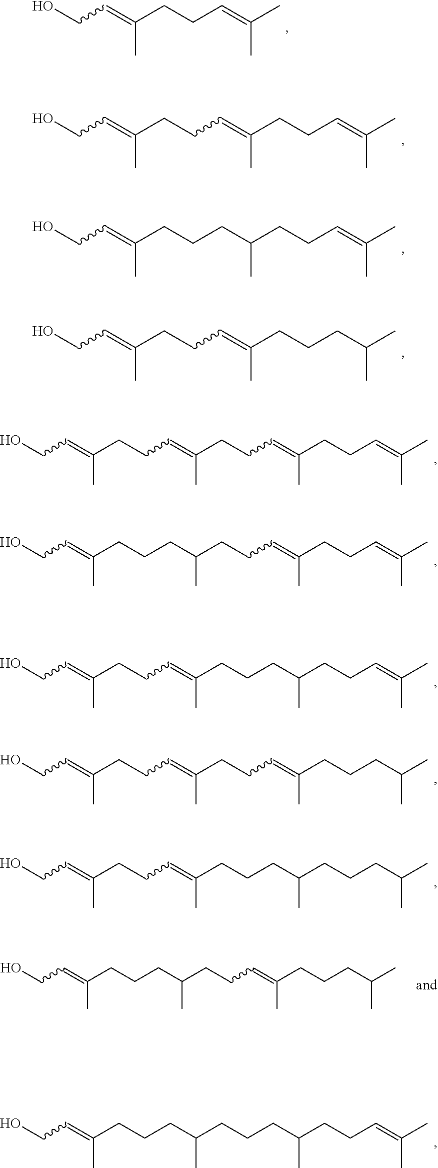

particularly selected from the group consisting of

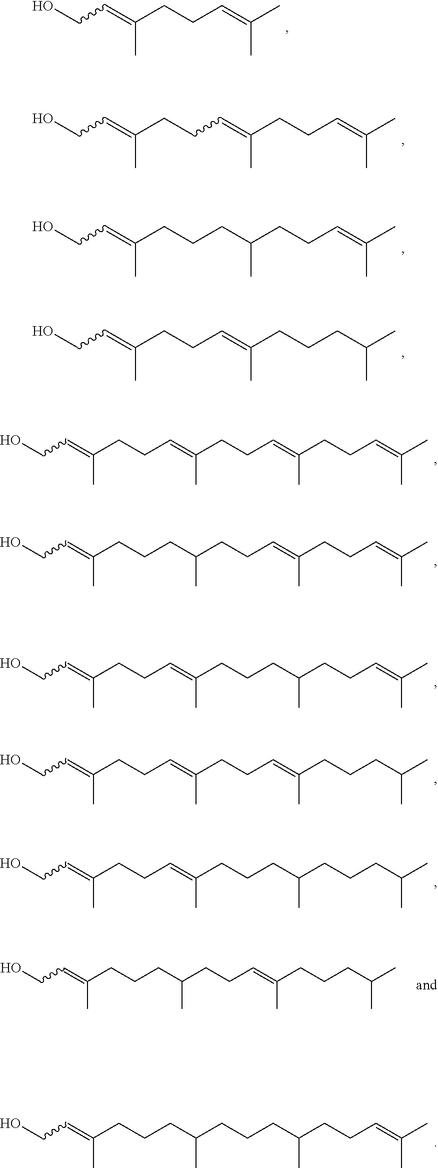

It is more preferred that all double bonds are in the E-configuration.

Most preferred compounds of formula (IIIa) are 3,7,11-trimethyldodeca-2,6,10-trien-1-ol, preferably (6E)-3,7,11-trimethyldodeca-2,6,10-trien-1-ol, also known as farnesol; and 3,7,11,15-tetramethylhexadeca-2,6,10,14-tetraen-1-ol, preferably (6E,10E)-3,7,11,15-tetramethylhexadeca-2,6,10,14-tetraen-1-ol, also known as geranylgeraniol. Particularly preferred are (2E,6E)-farnesol und (2E,6E,10E)-geranylgeraniol.

Examples for preferred compounds of the formula (IIIb) are compounds selected from the group consisting of

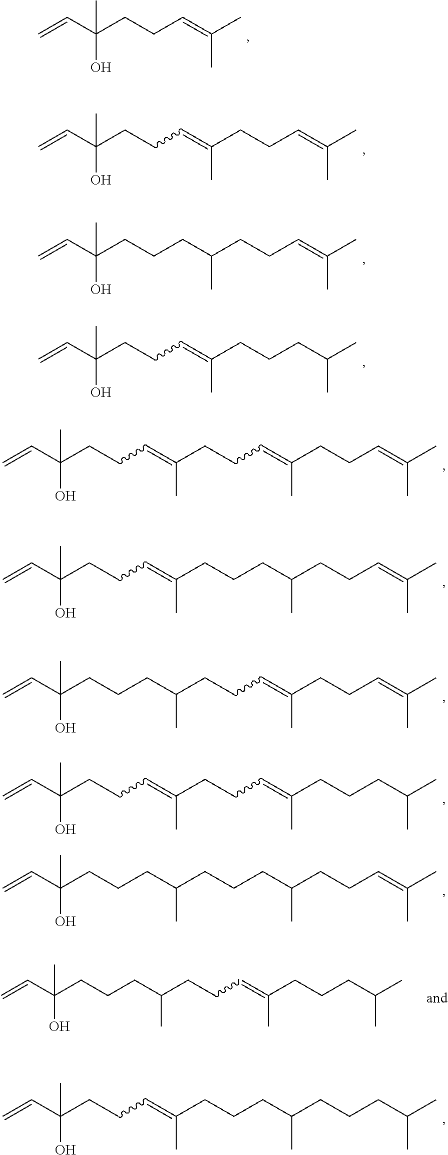

particularly selected from the group consisting of

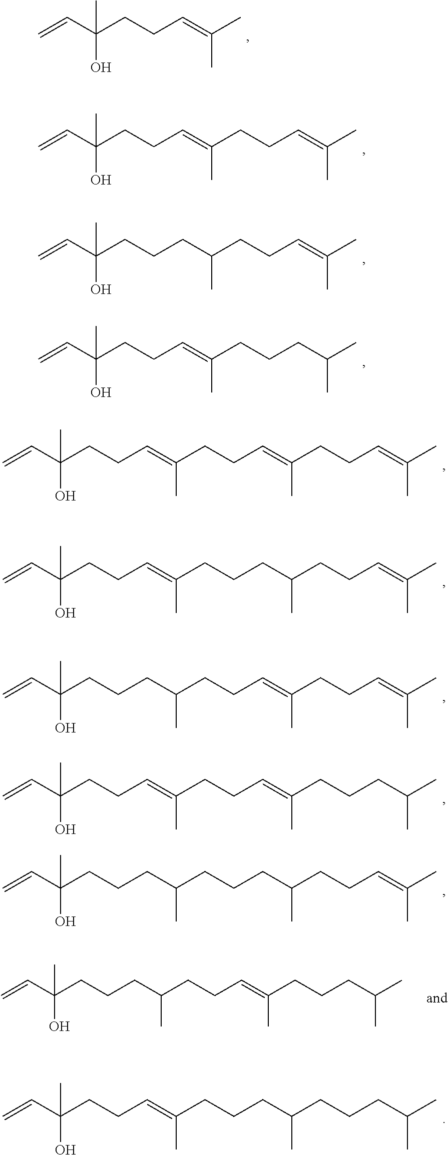

Most preferred compounds of formula (IIIb) are 3,7,11-trimethyldodeca-1,6,10-trien-3-ol, preferably (E)-3,7,11-trimethyldodeca-1,6,10-trien-3-ol, also known as nerolidol; and 3,7,11,15-tetramethylhexadeca-1,6,10,14-tetraen-3-ol, preferably (6E,10E)-3,7,11,15-tetramethylhexadeca-1,6,10,14-tetraen-3-ol, also known as geranyllinalool.

The compound of the formula (IIIa) and (IIIb), particularly geranyllinalool and geranylgeraniol, can either be prepared by known methods in industrial quantities or are also commercially available, for example from Sigma-Aldrich or from natural sources/bio-based sources, such as disclosed by John A. Hyatt et al., Organic Process Research and Development 2002, 6, 782-787.

### Acidic Catalyst

The compound of the formula (II) is reacted with a compound of the formula (IIIa) or (IIIIb) in the presence of a acidic catalyst which is either camphorsulfonic acid or BiCl₃ or Gd(OTf)₃ or Tm(OTf)₃ or Al(OTf)₃ or Y(OTf)₃ or Fe(OTf)₂.

It has been found that only a very small number of Brønsted or Lewis acids are suitable as acidic catalyst for the reaction of this invention.

In one embodiment, the acidic catalyst is camphorsulfonic acid. Camphorsulfonic acid as used in this document is camphor-10-sulfonic acid:

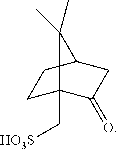

Preferably, camphorsulfonic acid is in its (+) form.

In another embodiment, the acid catalyst is BiCl₃.

In another embodiment, the acidic catalyst is the triflate of gadolinium or thulium or aluminum or yttrium or iron. Preferred triflates are Gd(OTf)₃ and Al(OTf)₃ and Fe(OTf)₂.

It is preferred that in above reaction, the molar ratio of the compound of formula (II) to the compound of formula (IIIa) or (IIIb) is in the range of between 3 and 1, preferably between 2.5 and 1.1, more preferably between 2.0 and 1.2.

Furthermore, it is preferred that the molar ratio of the compound of the formula (II) to the acidic catalyst is 0.001 mol % to 1 mol %, preferably 0.005% to 1 mol %, more preferably 0.02 to 1 mol %.

Said reaction is performed at a temperature of between 20° C. and 160° C., preferably between 80 and 120° C.

Furthermore, the reaction is typically performed at ambient pressure.

It is preferred that said reaction is performed in one embodiment in the presence of a solvent which is a hydrocarbon, preferably toluene, or a C₅₋₁₀ alkane, most preferred hexane or heptane.

It is preferred that said reaction is performed in another embodiment the presence of a solvent which is an organic carbonate, preferably a carbonate of the the formula (X)

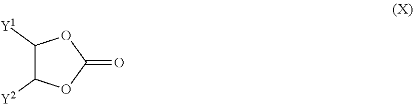

wherein Y¹ and Y² represent independently from each other either H or a methyl or ethyl group.

Preferably the organic carbonate is ethylene carbonate or propylene carbonate. It is also possible that mixtures of different carbonate of the formula (X) are used, particularly as binary or ternary mixture of ethylene carbonate and/or propylene carbonate and/or butylene carbonate, most preferably as a binary mixture of ethylene carbonate and propylene carbonate. It is preferred that the ratio of ethylene carbonate to propylene carbonate is between 20:80 to 80:20, particularly 25:75 to 75:25.

Preferably, the solvent of the formula is a carbonate solvent as commercialized by Huntsman under the trademark Jeffsol®, particularly the blends of ethylene carbonate with propylene carbonate Jeffsol® EC-75, Jeffsol® EC-50 and Jeffsol® EC-25.

In a further, even more preferred, embodiment said reaction is performed in the presence of a two-phase solvent mixture comprising at least one hydrocarbon and at least one organic carbonate. Said hydrocarbon is preferably toluene, or a C₅₋₁₀ alkane, most preferred hexane or heptane. Said organic carbonate is preferably a carbonate of the formula (X). Also in this embodiment the organic carbonate used can be a mixture of different carbonates of the formula (X), particularly as binary or ternary mixture of ethylene carbonate and/or propylene carbonate and/or butylene carbonate, most preferably as a binary mixture of ethylene carbonate and propylene carbonate. If mixtures of ethylene carbonate and propylene carbonate are used together with the hydrocarbon, it is preferred that the ratio of ethylene carbonate to propylene carbonate is between 20:80 to 80:20, particularly 25:75 to 75:25.

Most preferably, the reaction is performed in a two-phase solvent mixture of hexane and/or heptane with ethylene carbonate and/or propylene carbonate.

It is preferred that all bonds having dotted lines in formula (I) and (IIIa) and (IIIb) represent carbon-carbon double bonds.

The compound of the formula (I) can be oxidized to the compound of the formula (V) and cyclized to the compound of formula (VI).

Hence, in a further aspect, the present invention relates to a process of manufacturing the compound of the formula (VI)

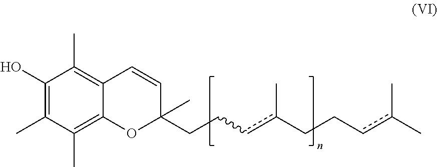

comprising the steps
a) manufacturing a compound of the formula (I) by a process as described above in great detail,

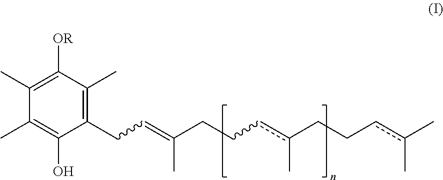

b) deprotecting the compound of the formula (I) to (I′) in case of R in the formula (I) is a phenol protecting group)

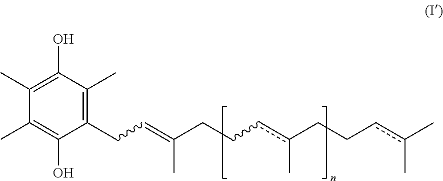

c) oxidizing compound of the formula

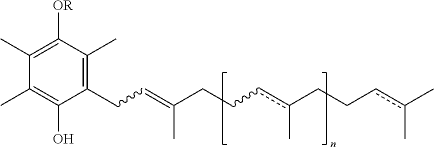

to the compound of the formula (V)

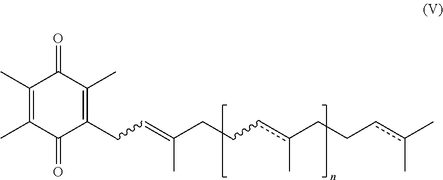

d) cyclization of compound of the formula (V) to the compound of the formula (VI) in the presence of a basic catalyst

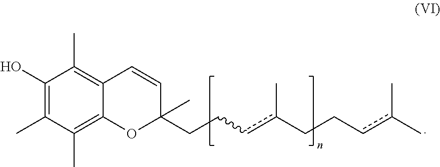

When the compound of the formula (I) has phenolic groups in a protected form, i.e. R═R′, the respective protection group needs to be removed by a deprotection reaction in a step b) to yield the respective deprotected compound of the formula (I′)

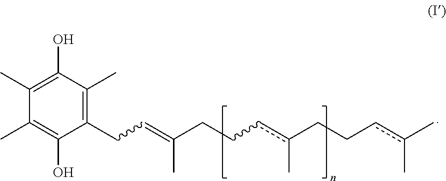

The conditions for said deprotection reaction in step b) depend on the kind of phenol protecting group and are known to the person skilled in the art. For example, esters can be easily deprotected under the influence of an acid or a base or acetals can be easily deprotected under the influence of acids.

Esters, such as acetate, are particularly cleaved to yield the respective unprotected phenol by lithium aluminum hydride.

The oxidation in step c) can be performed by the use of a suitable oxidizing agent, preferably with silver oxide in the presence of an acid, particularly acetic acid, or with oxygen or air in methanol.

In step d), the compound of the formula (V) is cyclized to the compound of the formula (VI) in the presence of a basic catalyst.

Said basic catalyst is preferably either an organic amine, preferably an organic tertiary amine, or a metal hydroxide or carbonate, particularly an organic tertiary amine or an alkali metal hydroxide.

It has been shown that the conjugated acids of said basic catalysts having a pKₐ of between 8.6 and 15.7, particularly of between 9 and 15.7, measured in water, are particularly suitable. This means that the basic catalyst has preferably a pK_{b} of between 5.4 and 0, particularly of between 5 and 0.

A few, non-limiting, examples of pkₐ of the corresponding acids for:

| Base | pka¹ |
|---|---|
| 1,5-diazabicyclo[4.3.0]non-5-ene (=DBN) | 13.42³ |
| 1,8-diazabicyclo[5.4.0]undec-7-ene (=DBU) | 12², 13.28³ |
| 1-azabicyclo[2.2.2]octane (=quinuclidine) | 11², 10.95³ |
| 4-(dimethylamino)pyridine (=DMAP) | 9.2², 9.7³ |
| sparteine | 9.45² |
| 1,4-diazabicyclo[2.2.2]octane (=DABCO) | 8.8², 8.82³ |
| NaOH | 15.7² |
| Na₂CO₃ | 10.4³ |
| morpholine⁴ | 8.36², 8.49³ |
| triethanolamine⁴ | 7.76³ |
| pyridine⁴ | 5.23², 5.23³ |

¹pka of the corresponding conjugated acid
²H. Ripin; D. A. Evans (2002). “pKₐ's of Nitrogen Acids” https://organicchemistrydata.org/hansreich/resources/pka/pka data/evans
³https://www.aatbio.com/data-sets/pka-and-pkb-reference-table
⁴basic catalysts which are less preferred (pkₐ < 8.6)

In one embodiment the basic catalyst is an organic amine, particularly selected from the group consisting of 4-dimethylaminopyridine (=DMAP), 1,8-diazabicyclo[5.4.0]undec-7-ene (=DBU), 1,5-diazabicyclo[4.3.0]non-5-ene (=DBN), 1,4-diazabicyclo[2.2.2]octane (=DABCO), 1-azabicyclo[2.2.2]octane (=quinuclidine) and sparteine, preferably from the group consisting of 4-dimethylaminopyridine (=DMAP), 1,8-diazabicyclo[5.4.0]undec-7-ene (=DBU) and 1-azabicyclo[2.2.2]-octane (=quinuclidine).

In another embodiment the basic catalyst is preferably a hydroxide or a carbonate, preferably a hydroxide, of an alkali metal or an earth alkali metal, particularly an alkali metal hydroxide. In this embodiment, most preferably, the basic catalyst is NaOH or KOH.

It is preferred that the ring closing step is performed in a hydrocarbon solvent, particularly in toluene.

In case a hydrocarbon solvent is used, the solvent is preferably used in such an amount that the solution with the compound of the formula (V) is between 0.05 and 5 molar, more preferred between 0.1 and 1 molar, in respect to the compound of the formula (V).

In case of water being present, it is preferred that the ring closure reaction is performed in a two-phase system, i.e. water phase and organic phase, particularly with a water and organic solvent phase

It is preferred that the basic catalyst is present in catalytical amounts, i.e. the basic catalyst is not present in stoichiometric amounts relative to the compound of the formula (V), but in significantly lower amounts, i.e. the molar ratio of the basic catalyst to the compound of the formula (V) is preferably 1:1′000 to 1:5, particularly 1:100 to 1:10.

The ring closing step is typically performed under stirring preferably at a temperature of between 40 and 200° C., preferably between 90 and 150° C., more preferably at the reflux temperature of the organic solvent when an organic solvent is used, and/or at a pressure of between 1 bara and 10 bara. It is further preferred that this reaction is performed under inert atmosphere, preferably under nitrogen.

The compound of the formula (VI) can be hydrogenated by means of a hydrogenation agent either compound of the formula (VIIII) (partial hydrogenation) or (IX) (complete hydrogenation)

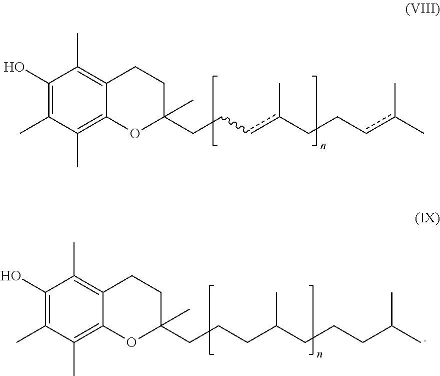

Hence, in a further aspect the present invention relates to a process of manufacturing a compound of the formula (VIII)

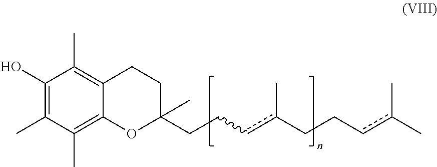

comprising the steps
i) manufacturing the compound of the formula (VI) by a process as described above in great detail

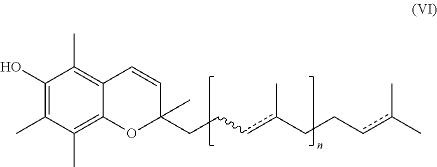

ii) partially hydrogenating the compound of the formula (VI) by means of a hydrogenating agent suitable for partial hydrogenation to yield the compound of the formula (VIII).

In the partial hydrogenation of step ii) only the carbon-carbon double bond in the ring is hydrogenated whereas the olefinic carbon-carbon double bonds are not hydrogenated (“partial hydrogenation”), so that the hydrogenation leads to the compound of the formula (VIII).

The hydrogenating agent used in step ii) is a hydrogenating agent which only hydrogenates the carbon-carbon double bond of the ring in formula (VIII). Particularly suitable as hydrogenating agent is sodium/ethanol such as described by Schudel, Mayer, Isler, Helv. Chim. Acta 46, 2517-2526 (1963), particularly in the last paragraph on page 2524.

This process particularly leads to α-tocotrienol, which has three double bonds in the side chain. α-Tocotrienol is an important compound in natural vitamin E.

Hence, in a further aspect the present invention relates to a process of manufacturing a compound of the formula (IX)

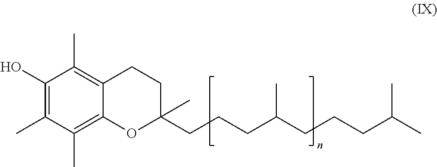

comprising the steps
i) manufacturing the compound of the formula (VI) by a process as described above in great detail

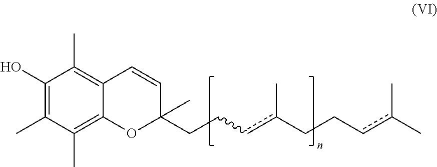

ii′) hydrogenating the compound of the formula (VI) by means of a hydrogenating agent to yield the compound of the formula (IX).

The hydrogenating agent used in step ii′) is a hydrogenating agent which hydrogenates all olefinic carbon-carbon double bond of the ring in formula (VI). Particularly suitable as hydrogenating agent is hydrogen in the presence of a transition metal from the groups 7, 8, 9 or 10, particularly selected form the group consisting of Pd, Pt, Rh, Ru, Mn, Fe, Co, and Ni, more preferably Pd.

The heterogenous transition metal catalyst is preferably a heterogenous supported transition metal catalyst.

Such hydrogenation is disclosed for example by Kabbe and Heitzer, Synthesis 1978, 12, 888-889.

In this embodiment, the transition metal is supported on a carrier, i.e. palladium is attached to/or deposited on a carrier. The carrier is a solid material.

Preferably said carrier is carbon or an inorganic carrier. Preferred inorganic carriers are oxides or carbonates. Preferred oxides are oxides of Si, Al, Ce, Ti or Zr, particularly of Al or Si. Particularly preferred are silicon dioxide, alumina and titanium dioxide and ceria.

In case the support is Ce, the preferred oxide is CeO₂. Preferably, the oxide of Al is Al₂O₃ and AlO(OH). Particularly preferred is Al₂O₃.

It is preferred to perform the hydrogenation under pressure, particularly under a hydrogen pressure of 2 to 20 bar. It is further preferred to perform the hydrogenation at a temperature between 0° C. and 100° C.

This process particularly leads to α-tocopherol, which has a completely saturated side chain. α-Tocopherol is an important compound in natural vitamin E.

The compound of the formula (I-A) is new. Hence, in a further aspect, the invention relates to a compound of the formula (I-A)

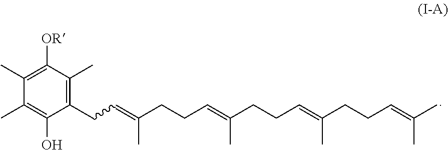

As already pointed out R′ represents a phenol protecting group and the wavy line represents a carbon-carbon bond which is linked to the carbon-carbon double bond which is either in the Z- or in the E-configuration. The protecting group has been discussed in great detail above. In this embodiment, the protecting group R′ is preferably an acetyl group.

It has been shown that an E/Z mixture is prepared from

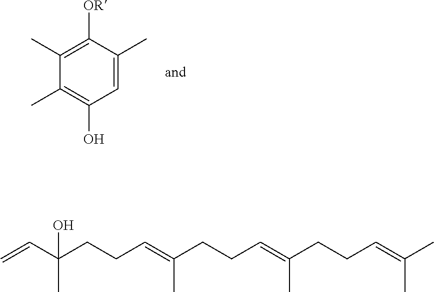

in the presence of according to the process discussed above in great detail.

The E/Z mixture can be separated by chromatography, if needed.

As this invention demonstrates, the compound of the formula (I) is a very suitable agent for the synthesis of compound of the formula (V′), (VI′) and (VIII′) respectively (IX′)

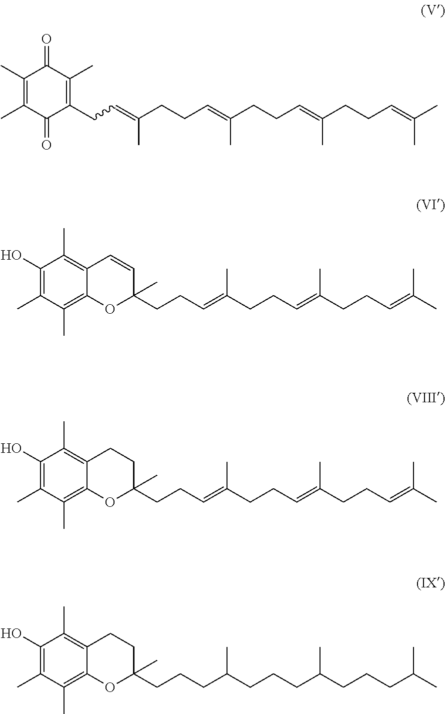 [SMILES: CC(C)=CCC/C(C)=C/CC/C(C)=C/CCC(C)=CCC1=C(C)C(=O)C(C)=C(C)C1=O ; CC(C)=CCC/C(C)=C/CC/C(C)=C/CCC1(C)C=Cc2c(C)c(O)c(C)c(C)c2O1 ; CC(C)=CCC/C(C)=C/CC/C(C)=C/CCC1(C)CCc2c(C)c(O)c(C)c(C)c2O1 ; Cc1c(C)c2c(c(C)c1O)CCC(C)(CCCC(C)CCCC(C)CCCC(C)C)O2]

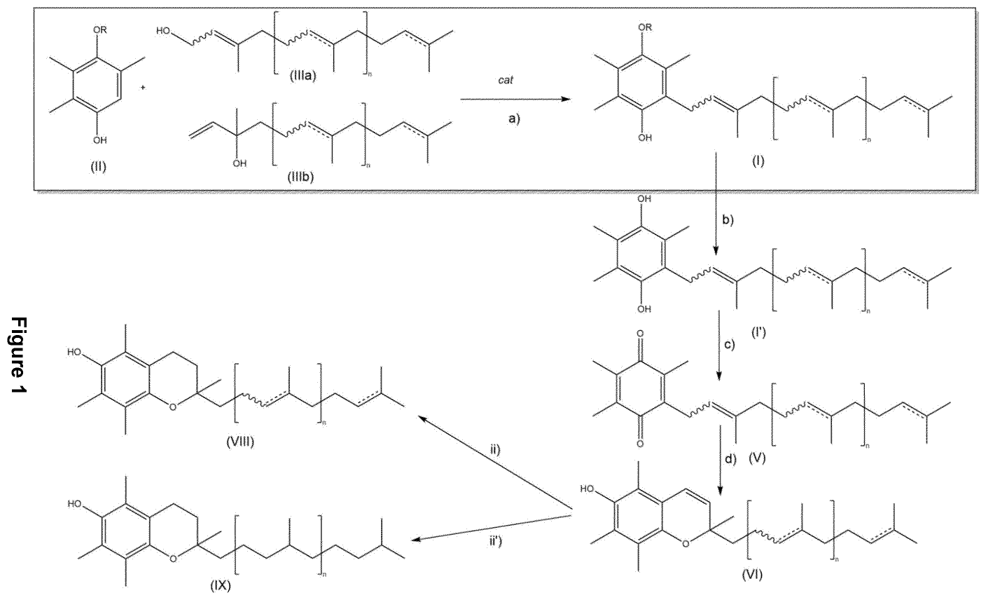

FIG. 1 shows schematically the processes discussed above in great detail, particularly, highlighted by the box, the process of manufacturing the compound of the formula (I) comprising a step of reacting a compound of the formula (II) with a compound of the formula (IIIa) or (IIIb) in the presence of an acidic catalyst (“cat”) being either Gd(OTf)₃ or Tm(OTf)₃ or Al(OTf)₃ or Y(OTf)₃ or Fe(OTf)₂ or camphorsulfonic acid or BiCl₃.

### EXAMPLES

The present invention is further illustrated by the following experiments.

### Experimental Series 1

Geranyllinalool (12.51 g (43 mmol)), 65 ml of a mixture ethylene carbonate/heptane (1.17/1 g/g) and 9.91 g (65 mmol) of 2,3,6-trimethylhydroquinone (TMHQ) have been added under stirring in the presence of the catalyst indicated in table 1 to yield 2,3,5-trimethyl-6-((2E,6E,10E)-3,7,11,15-tetramethyl-hexadeca-2,6,10,14-tetraen-1-yl)benzene-1,4-diol in conversion and yield as indicated in table 1.

TABLE 1
Different acidic catalysts for condensation
of TMHQ and geranyllinalool.

| | | Catalyst | Conversion | Yield |
|---|---|---|---|---|
| Example | Catalyst | [eq] | [%] | [%] |
| 1 | Camphorsulfonic acid¹ | 0.00025 | 100 | 32 |
| 2 | Camphorsulfonic acid | 0.0002 | 100 | 30 |
| 3 | Gd(OTf)₃ | 0.0000625 | 100 | 45 |
| 4 | Tm(OTf)₃ | 0.0001250 | 100 | 42 |

¹in all experiments of this document (+)-camphorsulfonic acid is used.

### Experimental Series 2

Geranylgeraniol (3.45 g (12 mmol)), 15 ml of a mixture ethylene carbonate/heptane (1.17/1 g/g) and 2.29 g (15 mmol) of 2,3,6-trimethylhydroquinone (TMHQ) have been added under stirring in the presence of the catalyst indicated in table 2 to yield 2,3,5-trimethyl-6-((2E,6E,10E)-3,7,11,15-tetramethyl-hexadeca-2,6,10,14-tetraen-1-yl)benzene-1,4-diol in conversion and yield as indicated in table 2.

TABLE 2
Different acidic catalysts for condensation
of TMHQ and geranylgeraniol.

| | | Catalyst | Conversion | Yield |
|---|---|---|---|---|
| Example | Catalyst | [eq] | [%] | [%] |
| 5 | Camphorsulfonic acid | 0.0005 | 100 | 23.1 |
| 6 | Al(OTf)₃ | 0.0002 | 100 | 38.6 |
| Ref. 1 | LiN(Tf)₂* | 0.1 | 39 | 5.0 |
| Ref. 2 | Cu/Zn/HCOOH** | 2.3 | 100 | 0 |

*LiN(Tf)₂ = Lithium bis((trifluoromethyl)sulfonyl)amide.
**Cu/Zn/HCOOH: Cu and Zn in formic acid (M. Kajiwara et al., Heterocyles, Vol. 14, No 12, 1995-1198, 1980).

### Experimental Series 3

Geranylgeraniol (3.42 g (12 mmol)), 15 ml of a mixture ethylene carbonate/heptane (1.17/1 g/g) and 2.91 g (15 mmol) of 2,3,6-trimethylhydroquinone-1-acetate have been added under stirring in the presence of the catalyst indicated in table 3 to yield 4-hydroxy-2,3,6-trimethyl-5-((2E,6E,10E)-3,7,11,15-tetramethylhexadeca-2,6,10,14-tetraen-1-yl)phenyl acetate in conversion and yield as indicated in table 3.

TABLE 3
Condensation of TMHQ-1 acetate and geranylgeraniol.

| | | Catalyst | Conversion | Yield |
|---|---|---|---|---|
| Example | Catalyst | [eq] | [%] | [%] |
| 7 | Camphorsulfonic acid | 0.001 | 100 | 20 |
| 8 | Camphorsulfonic acid | 0.0005 | 100 | 25 |
| 9 | Camphorsulfonic acid | 0.00025 | 100 | 35 |
| 10 | Camphorsulfonic acid | 0.000125 | 100 | 32 |

### Experimental Series 4

Geranyllinalool (12.51 g (43 mmol)), 65 ml of a mixture ethylene carbonate/heptane (1.17/1 g/g) and 16.54 g (65 mmol) of 2,3,6-trimethylhydroquinone-1-benzoate have been added under stirring in the presence of the catalyst indicated in table 4 to yield 4-hydroxy-2,3,6-trimethyl-5-((2E,6E,10E)-3,7,11,15-tetramethylhexadeca-2,6,10,14-tetraen-1-yl)phenyl benzoate in conversion and yield as indicated in table 4.

TABLE 4
Different acidic catalysts for condensation
of TMHQ-1 benzoate and geranylgeraniol.

| | | | Con- | | Selec- |
|---|---|---|---|---|---|
| | | Catalyst | version | Yield | tivity |
| Example | Catalyst | [eq] | [%] | [%] | [%] |
| 11 | Al(OTf)₃ | 0.0002 | >99 | 20 | 20 |
| 12 | Camphorsulfonic acid | 0.0002 | 89 | 24 | 27 |
| Ref. 3 | Bi(OTf)₃ | 0.0002 | 99 | 13 | 13 |

### Experimental Series 5

A solution of 2.19 g (11.3 mmol) of 2,3,6-trimethylhydroquinone-1-acetate and the amount of the catalyst indicated in table 5 in a mixture of 5.95 g of ethylene carbonate and 7.5 ml of n-heptane (ethylene carbonate/n-heptane 1.17/1 g/g) was heated to reflux under stirring. 2.2 g (7.5 mmol) of (all-E)-geranyllinalool were added. After removal of ethylene carbonate phase by extraction with heptane, 4-hydroxy-2,3,6-trimethyl-5-((6E,10E)-3,7,11,15-tetramethylhexadeca-2,6,10,14-tetraen-1-yl)phenyl acetate has been obtained in the yield as indicated in table 5.

TABLE 5
Different acidic catalysts for condensation of
TMHQ-1 acetate and (all-E)- geranyllinalool.

| | | Catalyst | Yield³ | Yield E¹ | Yield Z² |
|---|---|---|---|---|---|
| Example | Catalyst | [mol-%] | [%] | [%] | [%] |
| 13 | Gd(OTf)₃ | 1.0 | 31.8 | 29.0 | 2.8 |
| 14 | Fe(OTf)₂ | 0.4 | 31.3 | 28.5 | 2.8 |

¹Yield in the E-isomer
²Yield in the Z-isomer
³Yield Total yield (E + Z) determined by GC using internal standard.

### Experimental Series 6

A solution of 2.19 g (11.3 mmol) of 2,3,6-trimethylhydroquinone-1-acetate and the amount of the catalyst indicated in table 6 in a mixture of 5.95 g of ethylene carbonate and 7.5 ml of n-heptane (ethylene carbonate/n-heptane 1.17/1 g/g) was heated to reflux under stirring. 1.69 g (7.5 mmol) of (all-E)-farnesol or E-nerolidol were added. After removal of ethylene carbonate phase by extraction with heptane, 4-hydroxy-2,3,6-trimethyl-5-((6E)-3,7,11-trimethyldodeca-2,6,10-trien-1-yl)phenyl acetate has been obtained in the yield as indicated in table 6.

TABLE 6
Different acidic catalysts for condensation of TMHQ-1
acetate and E-nerolidol and (all E)-farnesol.

| | | | Catalyst | Yield¹ |
|---|---|---|---|---|
| Example | Alcohol | Catalyst | [mol-%] | [%] |
| 15 | E-nerolidol | Gd(OTf)₃ | 1.0 | 34.7 |
| 16 | E-nerolidol | Fe(OTf)₂ | 0.4 | 25.9 |
| 17 | (all E)-farnesol | Gd(OTf)₃ | 1.0 | 36.0 |
| 18 | (all E)-farnesol | Fe(OTf)₂ | 0.4 | 34.2 |

¹determined by GC using internal standard

1. A process of manufacturing the compound of the formula (I) 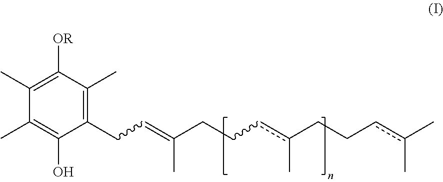
comprising a step of reacting a compound of the formula (II) with a compound of the formula (IIIa) or (IIIb) 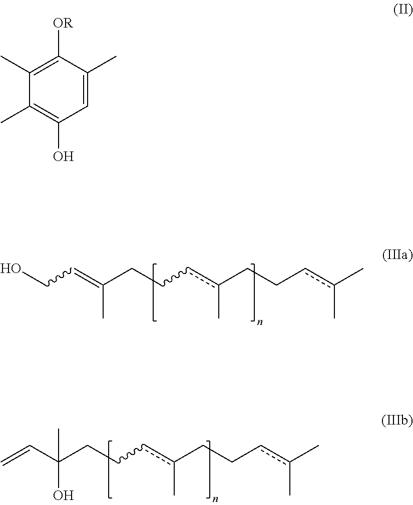
in the presence of an acidic catalyst being
either
Gd(OTf)₃ or Tm(OTf)₃ or Al(OTf)₃ or Y(OTf)₃ or Fe(OTf)₂
or
camphorsulfonic acid;
or
BiCl₃;
wherein
n=0 or 1 or 2 or 3 or 4 or 5 or 6 or 7 or 8 or 9 or 10 or 11 or 12;
R represents hydrogen or R′ which is a phenol protecting group;
OTf represents trifluoromethanesulfonate;
and
any bond having dotted line ( [symbol] ) represents independently from each other either a carbon-carbon single bond or a carbon-carbon double bond, with the proviso that at least one of the bonds having dotted lines represents a carbon-carbon double bond;
any wavy line represents independently from each other a carbon-carbon bond and which when linked to the carbon-carbon double bond is either in the Z- or in the E-configuration.

2. The process according to claim 1, wherein R′ is a phenol protecting group of the formula 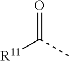
wherein R¹¹ is a C₁₋₁₅-alkyl or a fluorinated C₁₋₁₅-alkyl or a C₁₋₁₅-cycloalkyl or a C₇₋₁₅-aralkyl group, preferably a methyl or a benzyl group.

3. The process according to claim 1, wherein n=0 or 1 or 2, preferably 2.

4. The process according to claim 1, wherein all bonds having dotted lines in formula (I) and (IIIa) and (IIIb) represent carbon-carbon double bonds.

5. The process according to claim 1, wherein the molar ratio of the compound of formula (II) to the compound of formula (IIIa) or (IIIb) is in the range of between 3 and 1, preferably between 2.5 and 1.1, more preferably between 2.0 and 1.2.

6. The process according to claim 1, wherein the molar ratio of the compound of the formula (II) to the acidic catalyst is 0.001 mol % to 1 mol %.

7. The process according to claim 1, wherein the reaction is performed in the presence of a solvent which is a hydrocarbon, preferably toluene, or a C₅₋₁₀ alkane, most preferred hexane or heptane.

8. The process according to claim 1, wherein the reaction is performed in the presence of a solvent which is an organic carbonate, preferably a carbonate of the formula (X) 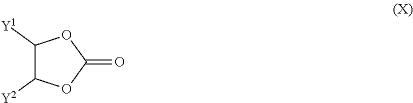
wherein Y¹ and Y² represent independently from each other either H or a methyl or ethyl group.

9. The process according to claim 1, wherein the reaction is performed in the presence of a two-phase solvent mixture comprising at least one hydrocarbon and at least one organic carbonate.

10. The process according to claim 1, wherein the reaction is performed at a temperature of between 20° C. and 160° ° C.

11. A process of manufacturing the compound of the formula (VI) 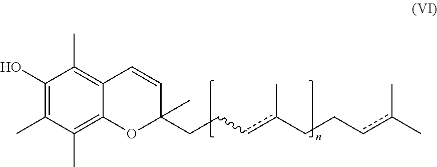
comprising the steps
a) manufacturing a compound of the formula (I) by a process according to claim 1, 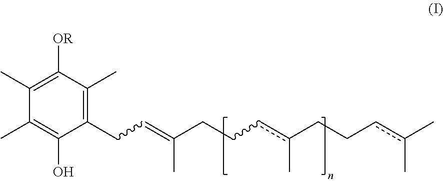
b) deprotecting the compound of the formula (I) to (I′) in case of R in the formula (I) is a phenol protecting group 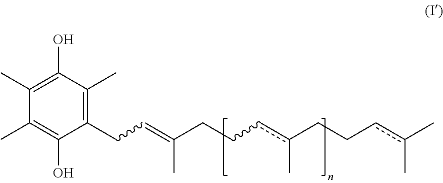
c) oxidizing compound of the formula 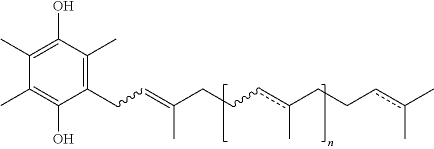
to the compound of the formula (V) 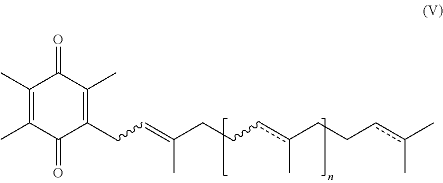
d) cyclization of compound of the formula (V) to the compound of the formula (VI) in the presence of a basic catalyst

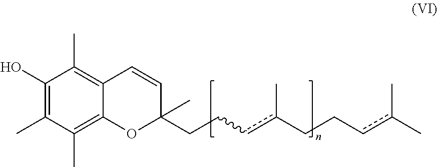

12. The process according to claim 11 wherein the basic catalyst in step d) is present in an molar ratio to the compound of the formula (V) of between 1:1′000 to 1:5, particularly 1:100 to 1:10.

13. The process of manufacturing a compound of the formula (VIII) 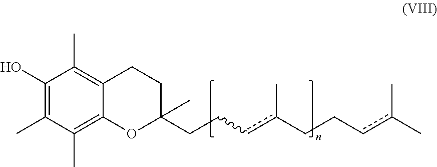
comprising the steps
i) manufacturing the compound of the formula (VI) by a process according to claim 11 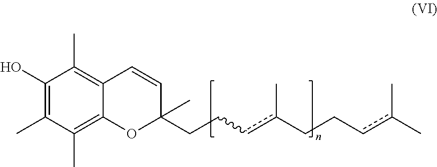
wherein any bond having dotted line ( [symbol] ) represents independently from each other either a carbon-carbon single bond or a carbon-carbon double bond; and
any wavy line represents independently from each other a carbon-carbon bond and which when linked to the carbon-carbon double bond is either in the Z or in the E-configuration;
ii) partially hydrogenating the compound of the formula (VI) by means of a hydrogenating agent suitable for partial hydrogenation to yield the compound of the formula (VIII).

14. The process of manufacturing a compound of the formula (IX) 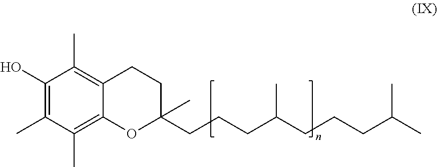
comprising the steps
i) manufacturing the compound of the formula (VI) by a process according to claim 11 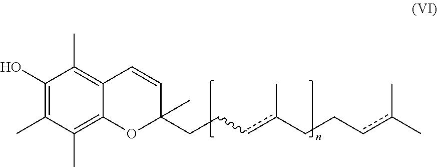
wherein any bond having dotted line ( [symbol] ) represents independently from each other either a carbon-carbon single bond or a carbon-carbon double bond; and
any wavy line represents independently from each other a carbon-carbon bond and which when linked to the carbon-carbon double bond is either in the Z or in the E-configuration;
ii′) hydrogenating the compound of the formula (VI) by means of a hydrogenating agent to yield the compound of the formula (IX).

15. The compound of formula (I-A) 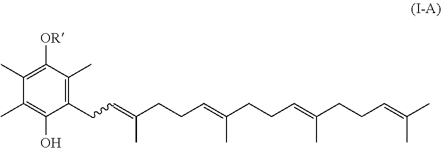
wherein R′ represents a phenol protecting group
and the wavy line represents a carbon-carbon bond linked to the carbon-carbon double bond which is either in the Z or in the E-configuration.
